(19) United States
(12) Patent Application Publication
(10) Pub. No.: US 2026/0174396 A1
(43) Pub. Date: Jun. 25, 2026

(54) AMBIENT NOISE DETECTION TO REDUCE HEART DISEASE EVENTS

(71) Applicant: Medtronic, Inc., Minneapolis, MN (US)

(72) Inventors: Bruce D. Gunderson, Plymouth, MN (US); Xusheng Zhang, Shoreview, MN (US); Andrew M. Freeman, Denver, CO (US)

(21) Appl. No.: 19/125,027

(22) PCT Filed: Sep. 26, 2023
(86) PCT No.: PCT/IB2023/059521

§ 371 Date: Apr. 28, 2025

Related U.S. Application Data
(60) Provisional application No. 63/381,452, filed on Oct. 28, 2022.

(51) Int. Cl. A61B 5/00

(52) U.S. Cl. CPC A61B 5/7275; A61B 5/4836; A61B 5/686

(57) ABSTRACT

A medical device system comprises a medical device comprising one or more sound sensors configured to generate a sound signal including noise experienced by a patient and processing circuitry. The processing circuitry is configured to determine, based on sound signal, one or more noise levels experienced by a patient, determine a heart disease risk for the patient based at least in part on the one or more noise levels, and generate an output corresponding to the heart disease risk to a computing device of the patient or another user.

This application claims the benefit of U.S. Provisional Patent Application Ser. No. 63/381,452, filed Oct. 28, 2022, the entire content of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Some types of devices may also be used to monitor one or more environmental conditions for the environment in which a patient finds themself. Such devices include implantable or wearable medical devices, as well as a variety of wearable health or fitness tracking devices. The physiological signals sensed by such devices include as examples, electrocardiogram (ECG) signals, electroencephalogram (EEG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

### SUMMARY

In general, the disclosure is directed to techniques for patient health monitoring and risk management. More particularly, the present disclosure describes techniques for recording (and in some examples, processing) ambient noise levels in a patient's environment using, for example, a medical device, e.g., an implantable medical device (IMD). The techniques further include determining a risk of heart disease based on the noise levels.

Excess noise in the environment of a patient may be associated with development or worsening of a variety of heart diseases, such as heart failure, arrhythmia, and coronary artery disease, or other patient conditions that are risk factors for heart disease, such as hypertension, diabetes, and obesity. Examples of conditions that may expose patients to excess noise include traffic and certain occupations. Some people may live or work in areas where they are frequently and/or chronically exposed to high environmental noise. Excess noise levels may activate stress responses, resulting in dysregulated cardiovascular function, cardiovascular tissue remodeling, and/or cell death.

The techniques of this disclosure may improve the functioning of a medical device system to monitor cardiovascular health of a patient. For example, a medical device of the system, e.g., an insertable cardiac monitor or other implantable medical device, may be configured to continuously and/or chronically monitor noise exposure of the patient, providing more comprehensive record of magnitude and impact of the patient's exposure to excessive noise than would otherwise be possible. In this manner, the system may be able to monitor the patient over time, e.g., on the order of months or years, without requiring direction or interventions by a clinician, the patient, or another person. Additionally, the system may provide a number of analyses to determine a risk level of cardiac disease based on application of criteria or predictive models, e.g., machine learning models, to the noise levels and, in some cases, other physiological data. In this manner, the techniques of this invention may allow a medical device system to more quickly and completely identify exposures to potentially harmful excess noise and communicate the resulting heart disease risk to the patient, the patient's clinicians, or other interested parties. In some cases, the communication of heart disease risk may advantageously facilitate, or even include recommendations/instructions to take actions to remediate excess noise exposure and/or lower heart disease risk level. Reduction or elimination of exposure to excess noise may help reduce or prevent heart disease or slow disease progression. The ability to continuously monitor noise levels and determine heart disease risk based on noise levels is lacking from conventional medical devices, and devices and techniques of this disclosure represent an improvement to the functioning of medical devices and systems to the benefit of patients by improving the ability of such devices and systems to monitor cardiac health.

In some examples, a medical device system comprises a medical device comprising one or more sound sensors configured to generate a sound signal including noise experienced by a patient. The medical device system further comprises processing circuitry configured to determine, based on the sound signal, one or more noise levels experienced by a patient, determine a heart disease risk for the patient based at least in part on the one or more noise levels, and generate an output corresponding to the heart disease risk to a computing device of the patient or another user.

In some examples, a method comprising determining, by processing circuitry of a medical device system including a medical device, based on sound signal generated by a sound sensor of the medical device, one or more noise levels experienced by a patient, determining, by the processing circuitry, a heart disease risk for the patient based at least in part on the one or more noise levels, and generating, by the processing circuitry, an output corresponding to the heart disease risk to a computing device of the patient or another user.

In some examples, a non-transitory computer readable storage medium comprises instructions that, when executed, cause processing circuitry of a medical device system to perform any of the methods described herein.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a block diagram illustrating an example system configured to detect noise levels experienced by a patient, and to respond to such detections, in accordance with one or more techniques of this disclosure.

FIG. 2 is a conceptual drawing illustrating example devices of the system of FIG. 1 sensing ambient noise levels, in accordance with one or more techniques described herein.

FIG. 3A is a perspective drawing illustrating an insertable cardiac monitor.

FIG. 3B is a perspective drawing illustrating another insertable cardiac monitor.

FIG. 4 is a block diagram illustrating an example configuration of a medical device that operates in accordance with one or more techniques of the present disclosure.

FIG. 5 is a block diagram illustrating an example configuration of a computing device that operates in accordance with one or more techniques of the present disclosure.

FIG. 6 is a block diagram illustrating a logical perspective of an example service operated by the example system of FIG. 5, in accordance with one or more examples of the present disclosure.

FIG. 7 is a flow diagram illustrating an example operation for generating an output of a heart disease risk caused by ambient noise in a patient's environment, in accordance with one or more techniques of the present disclosure.

FIG. 8 is a flow diagram illustrating an example operation for generating a noise profile for a patient over a time period, in accordance with one or more techniques described herein.

Like reference characters denote like elements throughout the description and figures.

### DETAILED DESCRIPTION

Evidence suggests an association between excess noise in the environment and cardiovascular disease. For example, individuals with bilateral high-frequency hearing loss may be around twice as likely to have coronary heart disease compared to the normal population. Individuals exposed to higher decibel traffic noises may have correspondingly higher risk of cardiovascular disease. According to the techniques of this disclosure, an implantable medical device (IMD) or other medical device may be configured to continuously determine ambient noise level in the surroundings, and the IMD or another component of a system may determine a heart disease risk level based on the noise levels.

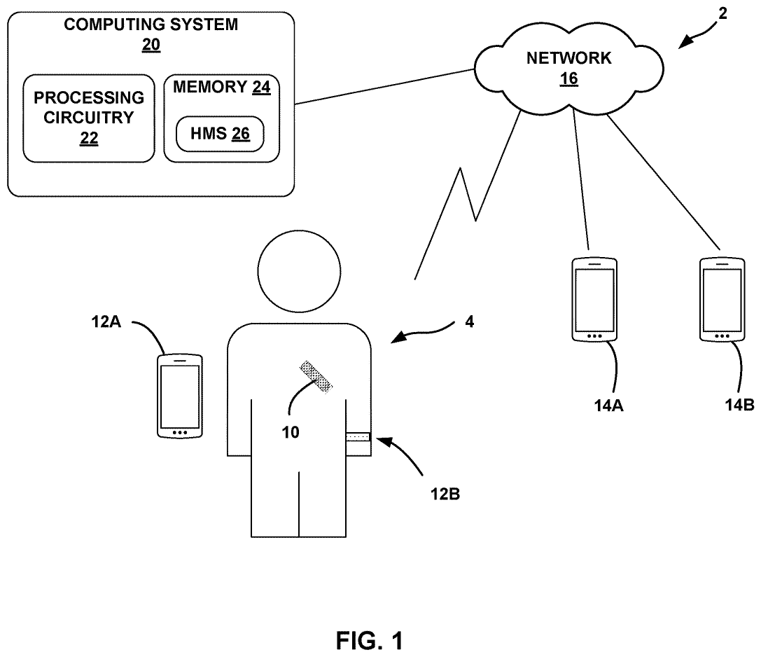

FIG. 1 is a conceptual diagram illustrating an environment of an example medical device system 2 in conjunction with a patient 4, in accordance with one or more techniques of this disclosure. System 2 includes an IMD 10. In some examples, IMD 10 is implanted outside of a thoracic cavity of patient 4 (e.g., subcutaneously in the pectoral location illustrated in FIG. 1). IMD 10 may be positioned near the sternum near or just below the level of patient 4's heart, e.g., at least partially within the cardiac silhouette. In some examples, IMD 10 takes the form of a LINQ™ Insertable Cardiac Monitor (ICM), available from Medtronic plc, of Dublin, Ireland. According to the techniques of this disclosure, IMD 10 includes one or more sound sensors to continuously record sound/noise in the environment of patient 4 over an extended period of time. IMD 10 may determine ambient noise levels to which patient 4 is exposed.

Although in one example IMD 10 takes the form of an ICM, in other examples, IMD 10 takes the form of any of a variety of implantable cardiac devices (ICDs) with intravascular or extravascular leads, such as pacemakers, intracardiac or extracardiac defibrillators, cardiac resynchronization therapy devices (CRT-Ds), neuromodulation devices, implantable sensors, or drug pumps, as examples. Additionally, or alternatively, techniques of this disclosure may be used to determine ambient noise levels to which patient 4 is exposed based on signals collected by one or more external medical devices such as patch devices, wearable devices (e.g., smart watches or fitness tracking devices), wearable sensors, or other external devices of patient 4 or in the environment of the patient, such as smart phone, smart home devices, other Internet of Things (IOT) devices, or any combination thereof.

Clinicians sometimes diagnose patients with medical conditions or monitor the progress of medical conditions based on one or more observed physiological signals collected by physiological sensors, such as electrodes, optical sensors, chemical sensors, temperature sensors, acoustic sensors, and motion sensors. In some cases, clinicians apply non-invasive sensors to patients during a clinic visit or hospital stay in order to sense one or more physiological signals while a patent is in a clinic for a medical appointment. Additionally, clinicians may ask patients questions, e.g., verbally or via a survey, to determine symptoms and environmental/behavioral factors that may be impacting the patients' health.

However, in some examples, physiological markers of a patient condition occur when the patient is outside the clinic. As such, in these examples, a clinician may be unable to observe the physiological markers needed to diagnose a patient with a medical condition. Additionally, it may be beneficial to monitor one or more patient parameters for an extended period of time (e.g., days, weeks, or months) so that the one or more parameters may be analyzed to identify a patient's unique physiological markers that accompany a symptom or medical condition. Furthermore, patients often provide incomplete information in response to queries regarding symptoms and environmental/behavior factors. In the example illustrated in FIG. 1, IMD 10 is implanted within patient 4 to continuously record one or more physiological signals of patient 4 over an extended period of time. Furthermore, according to the techniques of this disclosure, IMD 10 includes one or more sound sensors to continuously record sound/noise in the environment of patient 4 over an extended period of time, e.g., on the order of months or years. To continuously monitor noise in the environment of patient 4, IMD 10 may measure noise and determine noise levels, heart disease risks, or other metrics described herein, continuously on a periodic and/or triggered basis, without requiring intervention or direction from a clinician, patient 4, or another user.

In addition to sound sensors, IMD 10 may include any one or more electrodes, optical sensors, motion sensors (e.g., accelerometers), temperature sensors, chemical sensors, pressure sensors, or any combination thereof and any additional sensors that may be a part of IMD 10. Such sensors may sense one or more signals that indicate one or more physiological parameters of a patient. The one or more physiological parameters of the patient may be indicative of a patient condition, including a symptom or disease. Various features may be extracted from sensor signals, for example: the amount of deviation from a baseline; the timing of the deviation; absolute values corresponding to physiological parameters of a patient (e.g., a heart rate of 80 bpm) at a particular point in time.

IMD 10 may be configured to wirelessly communicate with one or more computing devices 12, such as patient computing devices 12A and 12B illustrated in FIG. 1. Computing devices 12 may be patient computing devices or clinician programming devices configured for use in settings such as a home, clinic, or hospital. Computing devices 12 may, in some examples, include a programmer, an external monitor, or a consumer device such as a smart phone, smart watch, or tablet.

As illustrated in FIG. 1, computing devices 12 may be coupled via a network 16 to a remote health monitoring system (HMS) 26. As illustrated in FIG. 1, HMS 26 may be implemented by processing circuitry 22 and memory 24 of a remote computing system, e.g., a cloud computing system, such as Carelink®, available from Medtronic plc, of Dublin, Ireland. Computing devices 12 may send data, such as data received from IMD 10 and data collected by computing devices 12, to computing system 20 via network 16. Computing devices 12 may communicate via near-field communication technologies (e.g., inductive coupling, near field communication (NFC), or other communication technologies operable at ranges less than 10-20 cm) and far-field communication technologies (e.g., the 802.11 or Bluetooth® specification sets, cellular network communications such as according to the 3G, 4G, or 5G protocols, or other communication technologies operable at ranges greater than near-field communication technologies).

Implementing HMS 26, processing circuitry 22 of computing system 20 may collect and process noise levels and other patient parameter data received from IMD 10 as described herein. Based on the analysis, processing circuitry 22 may determine risk of heart diseases, including risk of maladies contributing to heart diseases, such as risk heart failure, arrythmia, or hypertension. Processing circuitry 22 may generate outputs, such as messages, alerts, reports, network communications, or other communications, of noise levels, parameter data, risk levels, or other patient health metrics to patient 4 via computing devices 12, and other interested parties via their computing devices 14A and 14B (collectively, “computing devices 14”). Other interested parties may include clinicians, caregivers, and family members of patient 4. In some examples, the information provided by processing circuitry 22 may identify times and locations (e.g., determined based on global position system (GPS) data from computing device 12) at which patient 4 was exposed to excessive noise levels, so that the source of the noise levels may be identified and remediated or avoided.

In some examples, sounds sensors and one or more other sensors (e.g., electrodes, motion sensors, optical sensors, temperature sensors, or any combination thereof) of IMD 10 may sense one or more signals where each value of the signal represents a measurement, e.g., periodic measurement, at a respective interval of time. The plurality of values may represent a sequence of parameter values measured at a recurring time interval.

In another example, IMD 10 may perform a measurement in response to a patient notification that measurement should begin. In another example, IMD 10 may constantly perform parameter measurements. In this way, IMD 10 may be configured to track the patient condition more effectively as a patient need not be in a clinic for a parameter to be tracked, since IMD 10 is implanted within patient 4 and is configured to perform parameter measurements according to recurring or other time intervals without missing a time interval. In some examples, values may be measured, or measured values grouped, based on certain times of day, e.g., values measured during a window time during the day or a window of time during the night. In addition to time of day, IMD 10 may measure noise levels or other parameters in response to triggers, such as determining or receiving an indication that a physiological parameter or metric of patient condition has changed by more than a threshold from a baseline or recent average value, or determining based on a location indicated by computing device 12 (e.g., based on global positioning system (GPS) functionality of the computing device 12) that patient 4 has entered or exited a geofence.

In general, the techniques of this disclosure may be performed by processing circuitry of one or more devices of system 2, such as processing circuitry of one or more of IMD 10, computing device 12, or processing circuitry 22 of computing system 20.

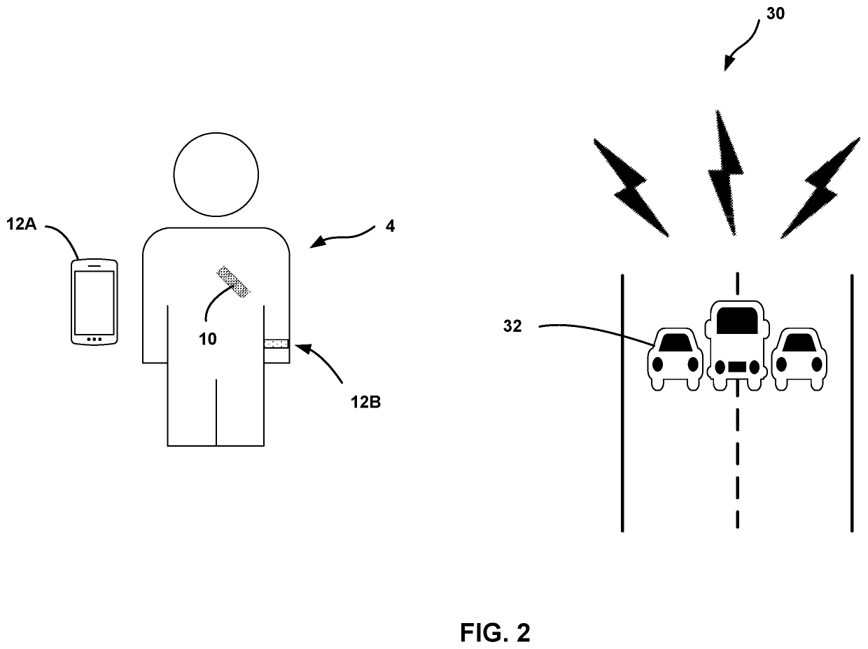

FIG. 2 is a conceptual drawing illustrating IMD 10 and computing device 12 of FIG. 1 sensing ambient noise levels, in accordance with one or more techniques described herein. IMD 10 includes one or more sound sensors configured to generate a signal in response to ambient noise 30, and thereby sense noise 30, in environment of patient 4. As will be described in greater detail below, IMD 10 processes the signal generated by the one or more sound sensors to determine noise levels to which patient 4 has been exposed.

FIG. 2 illustrates a source 32 of noise 30. Example noise sources 32 include traffic, machinery, aircraft, explosions or other incidents, and musical performances. In some examples, one or more components of system 2 may allow identification of noise sources 32, or conditions (e.g., situations) in which patient 4 was exposed to noise sources 32. For example, one or both of IMD 10 and computing devices 12 may be able to identify times of day and/or locations of patient 4 associated with noise levels. By presenting such information to patient 4 or other interested parties, system 2 may allow patient 4 to avoid noise sources in the future. By presenting such information to patient 4 or other interested parties, system 2 may allow patient 4 to take other actions to mitigate exposure to noise sources 32, e.g., when they cannot be avoided, such as wearing hearing protection equipment. In some cases, patient 4 may not appreciate that that their noise exposure is excessive or poses a health risk. In some cases, if patient 4 is exposed to longer-term environmental noise, e.g., over 30 days, actions may be recommended including adding sound insulation materials in the home or other environment in which patient 4 is exposed to noise sources, or moving to a less populous area. In some examples, through monitoring of noise levels (and in some cases determine heart disease risk) after action is taken to mitigate exposure to noise, system 2 may provide feedback to patient 4 or other users indicating the effectiveness of such actions.

In some examples, one or both of computing devices 12, or another computing device, may also include one or more sound sensors. In such examples, both IMD 10 and other device may be configured to determine noise levels based on their respective and sensing of the same noise 30, e.g., simultaneously. Processing circuitry of one or both of IMD 10 and computing device(s) 12 may calibrate the one or more sound sensors of IMD 10 or determination of noise levels by IMD 10 based on the signal sensed or noise levels determined by the computing device(s) 12. Such calibration may, for example, allow IMD 10 to determine a decibel level of noise 30 at the location of patient 4 outside patient 4 based on the signal sensed by the one or more sensors of IMD 10 inside patient 4. In some examples, one or both of computing devices 12 is configured to emit one or more sounds having known noise levels, e.g., decibel levels. Processing circuitry of system 2, e.g., of IMD 10 and/or computing device 12, may calibrate the one or more sound sensors of IMD 10 or determination of noise levels by IMD 10 based on the noise level of the signal emitted by computing device 12.

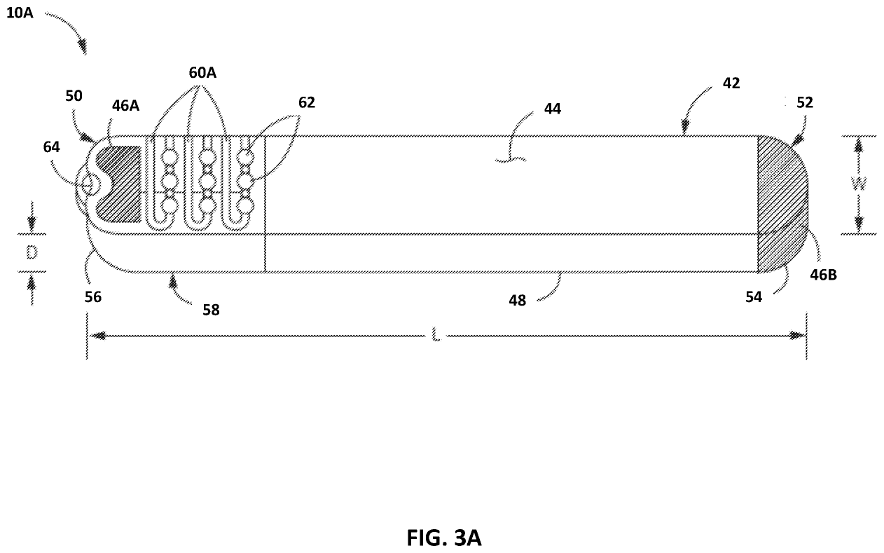

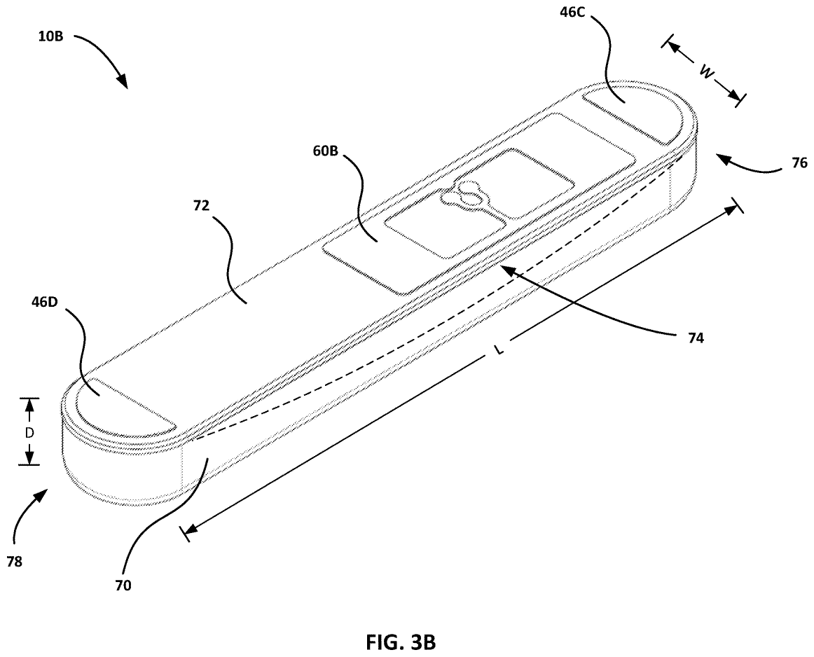

FIG. 3A is a perspective drawing illustrating an insertable cardiac monitor 10A, which may be an example configured of IMD 10 of FIG. 1 as an ICM. In the example shown in FIG. 3A, IMD 10A may be embodied as a monitoring device having housing 42, proximal electrode 46A and distal electrode 46B. Housing 42 may further comprise first major surface 44, second major surface 48, proximal end 50, and distal end 52. Housing 42 encloses electronic circuitry located inside the IMD 10A and protects the circuitry contained therein from body fluids. Housing 42 may be hermetically sealed and configured for subcutaneous implantation. Electrical feedthroughs provide electrical connection of electrodes 46A and 46B.

In the example shown in FIG. 3A, IMD 10A is defined by a length L, a width W and thickness or depth D and is in the form of an elongated rectangular prism wherein the length L is much larger than the width W, which in turn is larger than the depth D. In one example, the geometry of the IMD 10A-in particular a width W greater than the depth D-is selected to allow IMD 10A to be inserted under the skin of the patient using a minimally invasive procedure and to remain in the desired orientation during insertion. For example, the device shown in FIG. 3A includes radial asymmetries (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 3A, once inserted within the patient, the first major surface 44 faces outward, toward the skin of the patient while the second major surface 48 is located opposite the first major surface 44. In addition, in the example shown in FIG. 3A, proximal end 50 and distal end 52 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

Proximal electrode 46A is at or proximate to proximal end 50, and distal electrode 46B is at or proximate to distal end 52. Proximal electrode 46A and distal electrode 46B are used to sense electrocardiogram (ECG) signals thoracically outside the ribcage, which may be sub-muscularly or subcutaneously. ECG signals may be stored in a memory of IMD 10A, and data may be transmitted via integrated antenna 60A to another device, which may be another implantable device or an external device, such as computing device 12. In some example, electrodes 46A and 46B may additionally or alternatively be used for sensing any bio-potential signal of interest, which may be, for example, an electrogram (EGM), electroencephalogram (EEG), electromyogram (EMG), or a nerve signal, or for measuring impedance, from any implanted location.

In the example shown in FIG. 3A, proximal electrode 46A is at or in close proximity to the proximal end 50 and distal electrode 46B is at or in close proximity to distal end 52. In this example, distal electrode 46B is not limited to a flattened, outward facing surface, but may extend from first major surface 44 around rounded edges 54 and/or end surface 56 and onto the second major surface 48 so that the electrode 46B has a three-dimensional curved configuration. In some examples, electrode 46B is an uninsulated portion of a metallic, e.g., titanium, part of housing 42.

In the example shown in FIG. 3A, proximal electrode 46A is located on first major surface 44 and is substantially flat, and outward facing. However, in other examples proximal electrode 46A may utilize the three dimensional curved configuration of distal electrode 46B, providing a three dimensional proximal electrode (not shown in this example). Similarly, in other examples distal electrode 46B may utilize a substantially flat, outward facing electrode located on first major surface 44 similar to that shown with respect to proximal electrode 46A.

The various electrode configurations allow for configurations in which proximal electrode 46A and distal electrode 46B are located on both first major surface 44 and second major surface 48. In other configurations, such as that shown in FIG. 3A, only one of proximal electrode 46A and distal electrode 46B is located on both major surfaces 44 and 48, and in still other configurations both proximal electrode 46A and distal electrode 46B are located on one of the first major surface 44 or the second major surface 48 (e.g., proximal electrode 46A located on first major surface 44 while distal electrode 46B is located on second major surface 48). In another example, IMD 10A may include electrodes on both major surface 44 and 48 at or near the proximal and distal ends of the device, such that a total of four electrodes are included on IMD 10A. Electrodes 16A and 16B may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 3A, proximal end 50 includes a header assembly 58 that includes one or more of proximal electrode 46A, integrated antenna 60A, anti-migration projections 62, and/or suture hole 64. Integrated antenna 60A is located on the same major surface (i.e., first major surface 44) as proximal electrode 46A and is also included as part of header assembly 58. Integrated antenna 60A allows IMD 10A to transmit and/or receive data. In other examples, integrated antenna 60A may be formed on the opposite major surface as proximal electrode 46A, or may be incorporated within the housing 42 of IMD 10A. In the example shown in FIG. 3A, anti-migration projections 62 are located adjacent to integrated antenna 60A and protrude away from first major surface 44 to prevent longitudinal movement of the device. In the example shown in FIG. 3A, anti-migration projections 62 include a plurality (e.g., nine) small bumps or protrusions extending away from first major surface 44. As discussed above, in other examples anti-migration projections 62 may be located on the opposite major surface as proximal electrode 46A and/or integrated antenna 60A. In addition, in the example shown in FIG. 3A, header assembly 58 includes suture hole 64, which provides another means of securing IMD 10A to the patient to prevent movement following insertion. In the example shown, suture hole 64 is located adjacent to proximal electrode 46A. In one example, header assembly 58 is a molded header assembly made from a polymeric or plastic material, which may be integrated or separable from the main portion of IMD 10A.

FIG. 3B is a perspective drawing illustrating another IMD 10B, which may be another example configuration of IMD 10 from FIG. 1 as an ICM. IMD 10B of FIG. 3B may be configured substantially similarly to IMD 10A of FIG. 3A, with differences between them discussed herein.

IMD 10B may include a leadless, subcutaneously-implantable monitoring device, e.g., an ICM. IMD 10B includes housing having a base 70 and an insulative cover 72. Proximal electrode 46C and distal electrode 46D may be formed or placed on an outer surface of cover 72. Various circuitries and components of IMD 10B, e.g., described below with respect to FIG. 3, may be formed or placed on an inner surface of cover 72, or within base 70. In some examples, a battery or other power source of IMD 10B may be included within base 70. In the illustrated example, antenna 60B is formed or placed on the outer surface of cover 72, but may be formed or placed on the inner surface in some examples. In some examples, insulative cover 72 may be positioned over an open base 70 such that base 70 and cover 72 enclose the circuitries and other components and protect them from fluids such as body fluids. The housing including base 70 and insulative cover 72 may be hermetically sealed and configured for subcutaneous implantation.

Circuitries and components may be formed on the inner side of insulative cover 72, such as by using flip-chip technology. Insulative cover 72 may be flipped onto a base 70. When flipped and placed onto base 70, the components of IMD 10B formed on the inner side of insulative cover 72 may be positioned in a gap 74 defined by base 70. Electrodes 46C and 46D and antenna 60B may be electrically connected to circuitry formed on the inner side of insulative cover 72 through one or more vias (not shown) formed through insulative cover 72. Insulative cover 72 may be formed of sapphire (i.e., corundum), glass, parylene, and/or any other suitable insulating material. Base 70 may be formed from titanium or any other suitable material (e.g., a biocompatible material). Electrodes 46C and 46D may be formed from any of stainless steel, titanium, platinum, iridium, or alloys thereof. In addition, electrodes 46C and 46D may be coated with a material such as titanium nitride or fractal titanium nitride, although other suitable materials and coatings for such electrodes may be used.

In the example shown in FIG. 3B, the housing of IMD 10B defines a length L, a width W and thickness or depth D and is in the form of an elongated rectangular prism wherein the length L is much larger than the width W, which in turn is larger than the depth D, similar to IMD 10A of FIG. 3A. For example, the spacing between proximal electrode 46C and distal electrode 46D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 3B, once inserted subcutaneously within the patient, outer surface of cover 72 faces outward, toward the skin of the patient. In addition, as shown in FIG. 3B, proximal end 76 and distal end 78 are rounded to reduce discomfort and irritation to surrounding tissue once inserted under the skin of the patient. In addition, edges of IMD 10B may be rounded.

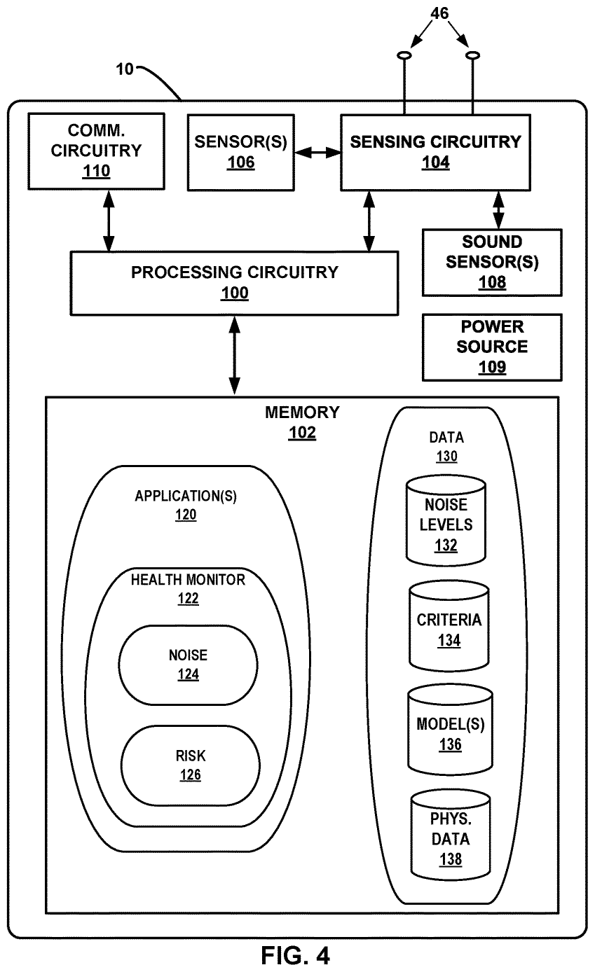

FIG. 4 is a block diagram illustrating an example configuration of IMD 10 in accordance with one or more techniques described herein. In the illustrated example, IMD 10 includes electrodes 46 (e.g., corresponding to any of electrodes 46A-46D), processing circuitry 100, memory 102, sensing circuitry 104, one or more patient parameter sensors 106, one or more sound sensors 108, and communication circuitry 110, which may be connected to an antenna 60 (FIGS. 3A and 3B). Processing circuitry 100 may be operatively coupled to memory 102, sensing circuitry 104, one or more patient parameter sensors 106, one or more sound sensors 108, and communication circuitry 110. Although the illustrated example includes two electrodes 46, IMDs including or coupled to more than two electrodes 46 may implement the techniques of this disclosure in some examples. IMD 10 further comprises a power source 109 to provide operational power for processing circuitry 100, memory 102, sensing circuitry 104, one or more patient parameter sensors 106, one or more sound sensors 108, and communication circuitry 110.

Processing circuitry 100 may include fixed function circuitry and/or programmable processing circuitry. Processing circuitry 100 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 100 may include multiple components, such as any combination of one or more microprocessors, one or more controllers, one or more DSPs, one or more ASICs, or one or more FPGAs, as well as other discrete or integrated logic circuitry. The functions attributed to processing circuitry 100 herein may be embodied as software, firmware, hardware or any combination thereof.

Sensing circuitry 104 may be coupled to electrodes 46, e.g., to sense electrical signals of the heart of patient 4, e.g., an ECG, as controlled by processing circuitry 100. In some examples, sensing circuitry 104 may include one or more filters and amplifiers for filtering and amplifying signals received from electrodes 46, patient parameter sensors 106, and/or sound sensors 108. Sensing circuitry 104 may include analog-to-digital conversion circuitry for converting the signals to digital samples for analysis by processing circuitry 100 and/or storage in memory 102.

The ECG sensed via electrodes 46 may represent one or more physiological electrical signals corresponding to the heart of patient 4. For example, the ECG may indicate ventricular depolarizations (QRS complexes including R-waves), atrial depolarizations (P-waves), ventricular repolarizations (T-waves), among other events. Information relating to the aforementioned events, such as time separating one or more of the events or the morphology of such events, may be applied for a number of purposes, such as to determine whether an arrhythmia is occurring, predict whether an arrhythmia is likely to occur, and/or determine a heart disease status of risk level of patient 4. In some examples, sensing circuitry 104 is configured to measure a tissue impedance signal via electrodes 46. The tissue impedance may be measured for a number of purposes, such as to determine a level of perfusion, edema, respiration rate, effort and pattern, and/or heart failure.

Sensors 106 may include an optical sensor. The optical sensor may, in some cases, include two or more light emitters and one or more light detectors. The optical sensor may perform one or more measurements in order to determine an oxygenation of the tissue of patient 4 or a blood pressure. Oxygen saturation and blood pressure may be indicative of one or more patient conditions, such as heart failure, hypertension, sleep apnea, or COPD, as examples. In some examples, sensors 106 include one or more accelerometers. An accelerometer may generate an accelerometer signal which reflects a measurement of a motion and/or posture of patient 4. In some cases, the accelerometer may collect a three-axis accelerometer signal indicative of patient 4's movements within a three-dimensional Cartesian space.

Sound (or acoustic) sensor(s) 108 may include a piezo-electric crystal or an accelerometer. Such sensors may be configured to generate a signal that varies with sound in the environment of patient 4. In some examples, sound sensor(s) 108, such as piezoelectric sensors, may generate the sound signal without requiring injection of current to the sensor, which may reduce the impact of continuous operation of the sound sensor on the power source of IMD 10. Sound sensor(s) 108 may be attached to an interior surface of, or otherwise within, a housing of IMD 10, although in other examples could be attached to an exterior surface of IMD 10 or coupled to IMD 10 via a lead.

Communication circuitry 110 may include any suitable hardware, firmware, software or any combination thereof for communicating with another device, such as computing device 12, another networked computing device, or another IMD or sensor. Under the control of processing circuitry 100, communication circuitry 110 may receive downlink telemetry from, as well as send uplink telemetry to computing device 12 or another device with the aid of an internal or external antenna, e.g., antenna 60. Antenna 60 and communication circuitry 110 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, memory 102 includes computer-readable instructions that, when executed by processing circuitry 100, cause IMD 10 and processing circuitry 100 to perform various functions attributed to IMD 10 and processing circuitry 100 herein. Memory 102 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Memory 102 may store, as examples, programmed values for one or more operational parameters of IMD 10. Memory 102 may also store data collected by IMD 10 for transmission to another device using communication circuitry 110 and/or further analysis by processing circuitry 100.

IMD 10 is an example of a medical device comprising one or more sound sensors 108 configured to generate a sound signal including noise experienced by patient 4. As illustrated in FIG. 4, memory 102 may store applications 120 and data 130 used by IMD 10, e.g., processing circuitry 100, to perform the techniques described in this disclosure related to determining health of patient 4 based on the sound signal.

For example, applications 120 may include a health monitor application 122. A noise component 124 of health monitor application 112 determine noise levels 132 experienced by patient 4 based on the sound signal. In some examples, each of noise levels 132 may be stored as data 130 in memory 102 in association with a time at which the noise level was measured. In some examples, noise levels 132 may be decibel levels of noise experienced by patient 4. Noise component 124 may apply a table or function from memory 102 to the sound signal to determine a decibel level, and the table or function may be calibrated as described above with respect to FIG. 2.

Noise component 124 may store each determined noise level, or only noise levels exceeding a threshold noise magnitude, as noise levels 132. Noise component 124 may store averages or other central tendency representations of noise magnitudes over periods of time as respective noise levels 132. Noise component 124 may store a maximum of noise magnitudes over periods of time as respective noise levels 132. In some examples, noise component 124 may store as, noise levels 132 for each of a plurality periods, integrations or summations of the sound signal during the period, or counts, percentages, sums, or other representations of amount of time during the period that the magnitude of the signal exceeded one or more thresholds. For example, noise component 124 may store as noise levels 132 values representing an amount or percentage of time the decibel level is between 60 and 80 decibels, and/or above 80 decibels during respective periods. Each period may be, for example, one or more seconds, one or more minutes, or one or more hours. The ability of noise component 124 to determine such metrics from an ambient sound signal as noise levels, a task infeasible by the human mind, may provide operational advantages in the ability of IMD 10 to monitor the cardiovascular health of patient 4.

Motion of patient 4 may introduce signal noise in the noise signal generated by sound sensor(s) 108, or otherwise confound the determination of noise levels 132 by noise component 124. In some examples, processing circuitry 100 may determine an activity level of patient 4, e.g., based on an accelerometer of parameter sensor(s) 106 and/or sound sensor(s) 108. Noise component 124 may configured to determine whether the activity level of patient 4 is below an activity threshold, and determine noise levels 132 based on the sound signal generated by sound sensor(s) 108 when the activity level of patient 4 is below the threshold.

In some examples, a risk component 126 of health monitor application 122 determines a heart disease risk of patient 4 based on noise levels 132, and generates an output, e.g., an alert or other message, of the heart disease risk to computing device 12 via communication circuitry 110. Risk component 126 may determine the heart disease risk by determining whether the noise levels 132 satisfy one or more noise exposure criteria 134. Noise exposure criteria 134 may include one or more threshold noise magnitudes and/or one or more threshold noise durations. In some examples, risk component 126 may associate different thresholds with different heart disease risk levels, such as mild, moderate, and extreme, or other risk level gradations/designations. In some examples, heart disease risk levels may be numerical values on a scale, e.g., from 1-10 or 1-100. In some examples, heart disease risk levels may be probabilities of patient 4 experiencing heart disease worsening, e.g., within some timeframe after noise exposure.

In some examples, different noise exposure criteria 134 may be associated with different time durations. For example, a first noise exposure criteria 134, e.g., threshold, may be compared to one or more noise levels 132 representing an instantaneous or short-term noise exposure, while a second noise exposure criteria 134, e.g., threshold, may be compared to noise levels 132 representing a longer-term or cumulative noise exposure, e.g., over one or more days, or a month.

In some examples, risk component 126 may be configured to apply noise levels 132, e.g., a time series of noise levels 132, as inputs to one or more machine learning models 136, which may output one or more values indicative of a probability or other heart disease risk level. Physiological data 138 may include values of physiological parameters determined based on signals sensed via electrodes 46 and parameter sensors 106, as discussed herein. In some examples, risk component 126 may apply physiological data as one or more additional inputs for the machine learning model(s) 136. In some examples, risk component 126 may apply techniques to determine heart disease risk based on noise levels 132 and, in some cases, other physiological data 138 similar to those described in commonly assigned U.S. Patent Application Publication No. 20120253207, titled “HEART FAILURE MONITORING,” which is incorporated herein by reference in its entirety. The techniques described by U.S. Patent Application Publication No. 20120253207 include applying evidence levels determined respectively from plurality of types of patient parameter data to Bayesian Belief Network or other probability model, which is an example of a machine learning model 136.

In some examples, noise levels 132 include a plurality of noise levels experienced by patient 4 over a period of time, from which risk component 126 may determine a noise profile. Risk component 126 may apply the profile to machine learning model 136, apply features of the profile to noise exposure criteria 134, or otherwise determine the heart disease risk based on the noise profile. The alert or other message output to computing device 12 regarding the heart disease risk may include the noise profile so that a user may also review and evaluate the noise profile. The computations employed by risk component 126 to determine heart disease risk based on noise levels, e.g., using the criteria or a machine learning model as described above, may not be performed by the human mind, and may provide advantages in the ability system 2 to monitor the cardiovascular health of patient 4.

In some examples, health monitoring application 122 is configured to determine that the patient is asleep based on physiological parameters sensed via electrodes 46 and/or parameter sensors 106, such as ECG signal, EEG signals, respiration signals, blood oxygenation signals, and blood pressure signals. Health monitoring application 122 may also be configured to determine one or more sleep metrics, e.g., indicative of depth or quality of sleep, while the patient is determined to be asleep based on these physiological parameters. Health monitoring application 122 may store the sleep metrics as physiological data 138 in memory 102.

Risk component 126 may correlate, e.g., by time, the one or more sleep metrics with one or more noise levels 132 experienced by patient 4, and determine the heart disease risk for the patient based at least in part on the correlation between the one or more sleep metrics and the one or more noise levels. Noise levels 132 were sufficient to disturb sleep may also be more likely to cause or worsen heart disease. To determine the heart disease risk, risk component 126 may be configured to apply features or metrics of the correlation to one or more criteria 134 or apply the time-correlated signals as inputs to one or more machine learning models 136.

The effect of noise on health of patient 4 may differ between daytime and nighttime. For example, an equal magnitude noise level may have a different physiological effect at night, e.g., due to a startle response, than during the day. For that reason, noise component 124 may group noise levels 132 into daytime (e.g., noon to 4 pm) or nighttime (e.g., midnight to 4 am) noise level groups, and calculate various statistical or other representations of noise levels 132 for daytime and nighttime. Boundaries of daytime and nighttime could be configurable by patient 4, a clinician, or other user via a computing device 12, 14. Furthermore, risk component 126 may determine heart disease risk based on noise levels 132 differently based on whether the noise levels 132 are daytime or nighttime noise levels. For example, risk component 126 may apply noise levels 132 to different thresholds (e.g., a daytime threshold and nighttime threshold) or other criteria 134, or different machine learning models 136, based on whether the noise levels 132 are daytime noise levels or nighttime noise levels.

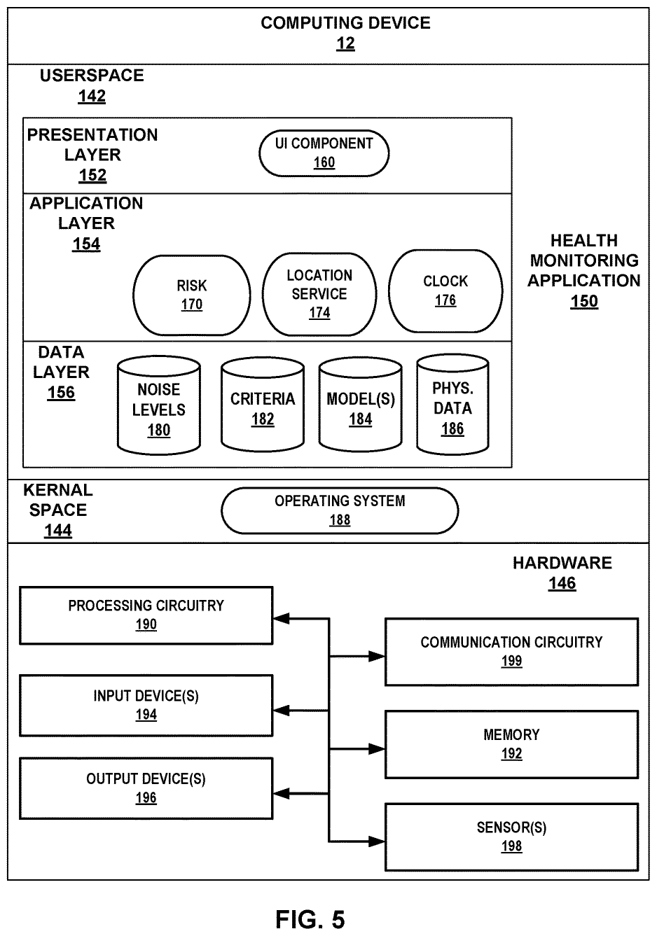

FIG. 5 is a block diagram illustrating an example configuration of a computing device 12, which may correspond to either (or both operating in coordination) of computing devices 12A and 12B of FIG. 1 that operates in accordance with one or more techniques of the present disclosure. In some examples, computing device 12 takes the form of a smartphone, a laptop, a tablet computer, a personal digital assistant (PDA), a smartwatch or other wearable computing device, or a smart speaker, smart home hub, or other internet of things (IoT) device.

As shown in the example of FIG. 5, computing device 12 may be logically divided into user space 142, kernel space 144, and hardware 146. Hardware 146 may include one or more hardware components that provide an operating environment for components executing in user space 142 and kernel space 144. User space 142 and kernel space 144 may represent different sections or segmentations of memory, where kernel space 144 provides higher privileges to processes and threads than user space 142. For instance, kernel space 144 may include operating system 188, which operates with higher privileges than components executing in user space 142.

As shown in FIG. 5, hardware 146 includes processing circuitry 190, memory 192, one or more input devices 194, one or more output devices 196, one or more sensors 198, and communication circuitry 199. Although shown in FIG. 5 as a stand-alone device for purposes of example, computing device 12 may be any component or system that includes processing circuitry or other suitable computing environment for executing software instructions and, for example, need not necessarily include one or more elements shown in FIG. 5.

Processing circuitry 190 is configured to implement functionality and/or process instructions for execution within computing device 12. For example, processing circuitry 190 may be configured to receive and process instructions stored in memory 192 that provide functionality of components included in kernel space 144 and user space 142 to perform one or more operations in accordance with techniques of this disclosure. Examples of processing circuitry 190 may include, any one or more microprocessors, controllers, GPUs, TPUs, DSPs, ASICs, FPGAs, or equivalent discrete or integrated logic circuitry.

Memory 192 may be configured to store information within computing device 12, for processing during operation of computing device 12. Memory 192, in some examples, is described as a computer-readable storage medium. In some examples, memory 192 includes a temporary memory or a volatile memory. Examples of volatile memories include random access memories (RAM), dynamic random access memories (DRAM), static random access memories (SRAM), and other forms of volatile memories known in the art. Memory 192, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 194 of computing device 12 may receive input, e.g., from patient 4 or another user. Examples of input are tactile, audio, kinetic, and optical input. Input devices 194 may include, as examples, a mouse, keyboard, voice responsive system, camera, buttons, control pad, microphone, presence-sensitive or touch-sensitive component (e.g., screen), or any other device for detecting input from a user or a machine. In some examples, a microphone of input devices 194 may act as a sound sensor to sense ambient noise at the same time as sound sensor(s) 108 of IMD 10 for purposes of calibrating the determination of noise levels 132 by IMD 10 as described above.

One or more output devices 196 of computing device 12 may generate output, e.g., to patient 4 or another user. Examples of output are tactile, haptic, audio, and visual output. Output devices 196 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output. In some examples, a speaker of output devices 196 may generate a predetermined noise for purposes of calibrating the determination of noise levels 132 by IMD 10 as described above.

One or more sensors 198 of computing device 12 may sense physiological parameters or signals of patient 4. Sensor(s) 198 may include electrodes, accelerometers (e.g., 3-axis accelerometers), an optical sensor, impedance sensors, temperature sensors, pressure sensors, heart sound sensors (e.g., microphones), and other sensors, and sensing circuitry (e.g., including an ADC), similar to those described above with respect to IMD 10 and FIG. 4.

Communication circuitry 199 of computing device 12 may communicate with other devices by transmitting and receiving data. Communication circuitry 199 may include a network interface card, such as an Ethernet card, an optical transceiver, a radio frequency transceiver, or any other type of device that can send and receive information. For example, communication circuitry 199 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 5, health monitoring application 150 executes in user space 142 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, a risk component 170, location service 174, and clock 176. Risk component 170 may determine heart disease risk based on noise levels 180 received from IMD 10 via communication circuitry 199. For example, risk component 170 may determine the heart disease risk based on comparison of the noise levels 180 to one or more criteria 182 and/or application of the one or more noise levels 180 to one or more machine learning models 184 in the manner described above with respect to IMD 10 and FIG. 4. In some examples, in addition to noise levels 180, risk component 170 may apply physiological data 186 of patient 4 to the criteria 182 and/or model(s) 184 to determine the heart disease risk in the manner described above with respect to IMD 10 and FIG. 4.

Physiological data 186 may include data collected by IMD 10 as described above, and received via communication circuitry 199. In some examples, physiological data 186 may include data collected by processing circuitry 190 via input devices 194 and sensor(s) 198. As examples, sensed data from computing device 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns. In some examples, the patient data may include responses to queries posed by health monitoring application 150 regarding the condition of patient 4 via output devices 196, input by patient 4 via input devices 194.

Location service 174 may determine the location of computing device 12 and, thereby, the presumed location of patient 4. Location service 178 may use GPS data, multilateration, and/or any other known techniques for locating computing devices. Clock 176 may generate data indicating the time of day associated with the locations of patient 4. Furthermore, based on time-stamp data from IMD 10, processing circuitry 190 may associate noise levels 180 received from IMD 10 with the locations and times of day. In conjunction with the heart disease risk information, output devices 196 may present noise levels 180 determined to be excessive or causative of the heart disease risk and their associated locations and times of day. Based on that information, patient 4 or another interested user may identify causes of the noise exposure.

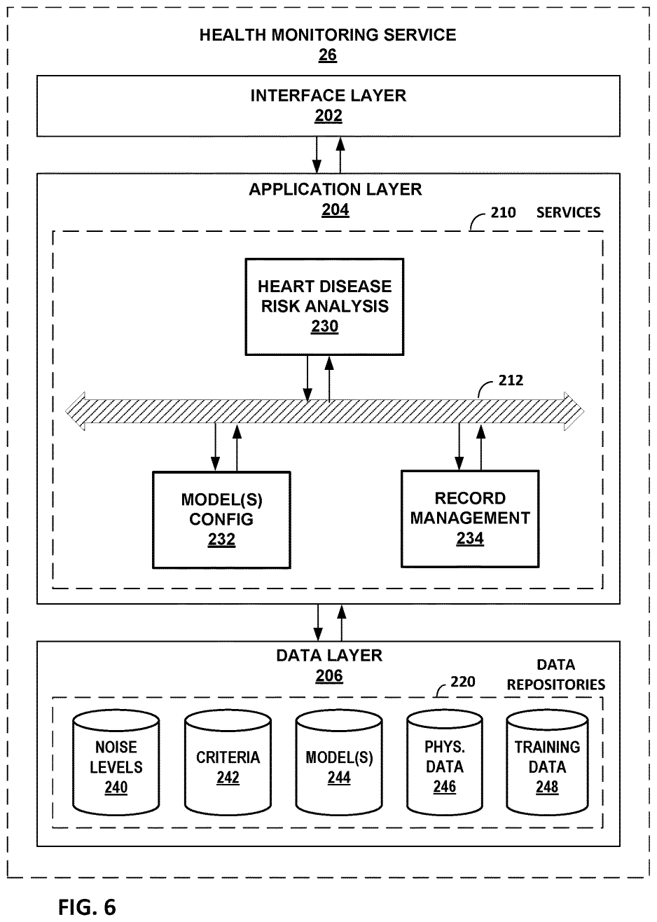

FIG. 6 is a block diagram illustrating a logical perspective of health monitoring service (HMS) 26 operated by computing system 20 (FIG. 1). An example implementation typically involves various hardware/software components operating on processing circuitry 22 of computing system 20 (FIG. 1) and generally configured to be network-accessible for transmitting/receiving various data to/from patient medical devices (e.g., IMD 10), their local devices (e.g., computing device(s) 12), computing devices 14 of other users, and/or the like.

FIG. 6 provides an operating perspective of HMS 26 when hosted as a cloud-based platform. In the example of FIG. 6, components of HMS 26 are arranged according to multiple logical layers that implement the techniques of this disclosure. Each layer may be implemented by one or more modules comprised of hardware, software, or a combination of hardware and software.

Computing devices, such as computing devices 12 and 14 of FIG. 1, may operate as clients that communicate with HMS 26 via interface layer 202. The computing devices typically execute client software applications, such as desktop applications, mobile applications, and web applications, e.g., health monitoring application 150. Interface layer 202 represents a set of application programming interfaces (API) or protocol interfaces presented and supported by HMS 26 for the client software applications. Interface layer 202 may be implemented with one or more web servers.

As shown in FIG. 6, HMS 26 also includes an application layer 204 that represents a collection of services 210 for implementing the functionality ascribed to HMS 26 herein. Application layer 204 receives information from client applications, e.g., noise levels determined by IMD 10 and, in some cases, associated locations of patient 4 and times determined by computing device(s) 12, and stores them as noise levels 240. Application layer 204 may similarly receive physiological data 246 from IMD 10 and computing device(s) 12. Application layer 204 processes the information according to one or more of the services 210 to respond to the information. Application layer 204 may be implemented as one or more discrete software services 210 executing on one or more application servers, e.g., physical or virtual machines. That is, the application servers provide runtime environments for execution of services 210. In some examples, the functionality of interface layer 202 as described above and the functionality of application layer 204 may be implemented at the same server. Services 210 may communicate via a logical service bus 212. Service bus 212 generally represents a logical interconnection or set of interfaces that allows different services 210 to send messages to other services, such as by a publish/subscription communication model.

Data layer 206 of HMS 26 provides persistence for information in HMS 26 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 6, each of services 230-234 is implemented in a modular form within HMS 26. Although shown as separate modules for each service, in some examples the functionality of two or more services may be combined into a single module or component. Each of services 230-234 may be implemented in software, hardware, or a combination of hardware and software. Moreover, services 230-234 may be implemented as standalone devices, separate virtual machines or containers, processes, threads or software instructions generally for execution on one or more physical processors.

Heart disease risk analysis service 230 may perform any techniques described herein for determining heart disease risk based on application of noise levels 240 (and in some cases physiological data 246) to one or more criteria 242 and/or machine learning models 244. Machine learning model configuration service 232 may train, validate, and otherwise configure machine learning models 244 using training data 248. Training data 248 may include numerous sets of noise levels (and in some cases time corresponding physiological data) from various subjects, that has been labeled with a heart disease risk level. Example machine learning techniques that may be employed to generate one or more models 244 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

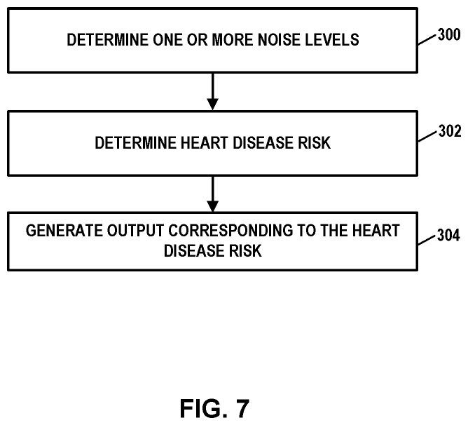

FIG. 7 is a flow diagram illustrating an example operation for generating an output to a user of heart disease risk of a patient caused by ambient noise in a patient's environment, in accordance with one or more techniques of the present disclosure. The example operation of FIG. 7 is described as being performed by processing circuitry, which may comprise one or more of processing circuitry 100 of IMD 10, processing circuitry 190 of computing device 12, or processing circuitry 22 of computing system 20.

According to the example of FIG. 7, the processing circuitry determines one or more noise levels based on a sound signal generated by one or more sound sensors of IMD 10 (300). The noise levels may be magnitudes (e.g., in decibels), representative values determined from such magnitudes over time periods, representations of time that the magnitudes where over one or more thresholds during time periods, or any other noise level values described herein. The processing circuitry determines a heart disease risk based on the noise levels (302). The risk may be a numerical or qualitative value of the magnitude of risk, or a probability of developing or worsening of a heart disease or contributing condition, such as heart failure, arrhythmia, or hypertension, as described herein. The processing circuitry may generate an output, e.g., an alert or other report, of the heart disease risk (304). The output corresponding to the heart disease risk may be via communication with a computing device 12 of patient 4 or computing device 14 of another user.

In some examples, the processing circuitry may create a daily graph of time above a decibel threshold, where the decibel threshold corresponds to significantly noise environments. One or more computing devices configured to display the graph and/or other information related to noise exposure and heart disease risk. In some examples, additional or alternative graphs may include representative values of noise levels for periods graphed over a longer time scale.

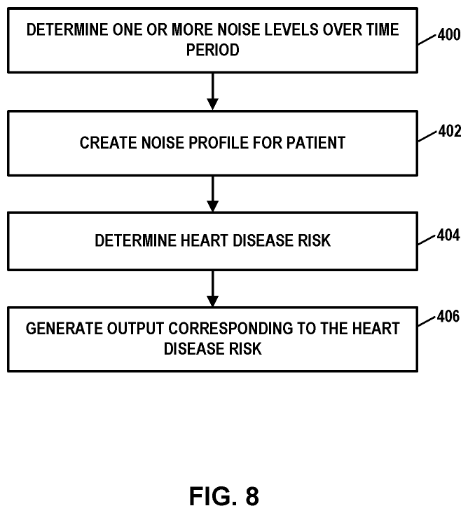

FIG. 8 is a flow diagram illustrating an example operation for generating a noise profile for a patient over a time period, in accordance with one or more techniques described herein. The example operation of FIG. 8 is described as being performed by processing circuitry, which may comprise one or more of processing circuitry 100 of IMD 10, processing circuitry 190 of computing device 12, or processing circuitry 22 of computing system 20.

According to the example of FIG. 8, the processing circuitry determines one or more noise levels over a time period, such as one or more hours or a day, based on a sound signal generated by one or more sound sensors of IMD 10 (400). The processing circuitry creates a noise profile based on the noise levels (402), and determines a heart disease risk for the patient based on the profile (404). The profile may characterize amounts of noise exposure over the time period based on the time sequence of noise levels during the period. The processing circuitry may apply the profile to a machine learning model as a time series of nose levels, or apply features of the profile to noise exposure criteria, to determine the heart disease risk based on the noise profile. The processing circuitry may generate an output corresponding to the heart disease risk, e.g., via communication with a computing device 12 of patient 4 or computing device 14 of another user (304). In some cases, the output, e.g., alert or report, may include a representation, e.g., graph, of the profile.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

The following examples are a non-limiting list of clauses in accordance with one or more techniques of this disclosure.

Example 1. A medical device system comprising: a medical device comprising one or more sound sensors configured to generate a sound signal including noise experienced by a patient; and processing circuitry configured to: determine, based on the sound signal, one or more noise levels experienced by a patient; determine a heart disease risk for the patient based at least in part on the one or more noise levels; and generate an output corresponding to the heart disease risk to a computing device of the patient or another user.

Example 2. The medical device system of Example 1, wherein to determine the one or more noise levels, the processing circuitry is configured to determine a plurality of noise levels experienced by the patient over a time period, and wherein the processing circuitry is further configured to: create a noise profile for the patient based on the plurality of noise levels determined during the time period, wherein the noise profile comprises at least the plurality of noise levels determined and a corresponding time stamp for each of the plurality of noise levels; and determine the heart disease risk for the patient based at least in part on the noise profile.

Example 3. The medical device system of Example 2, wherein the processing circuitry is further configured to output the noise profile for the patient for display on the computing device.

Example 4. The medical device system of any one or more of Examples 1-3, wherein the one or more noise levels are determined during one or more regularly occurring, predetermined time periods.

Example 5. The medical device system of Example 4, wherein the one or more regularly occurring, predetermined time periods comprise one or both of a nighttime period and a daytime period.

Example 6. The medical device system of Example 5, wherein to determine the heart disease risk the processing circuitry is configured to: compare the one or more noise levels during the nighttime period to a first one or more criteria; and compare the one or more noise levels during the daytime period to a second one or more criteria that are different than the first one or more criteria.

Example 7. The medical device system of any one or more of Examples 1-6, wherein the processing circuitry is further configured to: determine that an activity level of the patient is below a threshold; and determine the one or more noise levels experienced by the patient based on the sound signal generated when the activity level of the patient is below the threshold.

Example 8. The medical device system of Example 7, wherein the medical device comprises an accelerometer configured to generate a motion signal of the patient, wherein the processing circuitry determines the activity level based on the motion signal.

Example 9. The medical device system of any one or more of Examples 1-8, wherein the processing circuitry is further configured to identify one or more noise conditions causing the one or more noise levels experienced by the patient.

Example 10. The medical device system of Example 9, wherein to identify the one or more noise conditions, the processing circuitry is further configured to determine locations of the patient associated with the noise levels via the global positioning system data.

Example 11. The medical device system of Example 9 or 10, further comprising a clock, wherein to identify the one or more noise conditions, the processing circuitry is further configured to determine times of day associated with the noise levels via the clock.

Example 12. The medical device system of any one or more of Examples 1-11, wherein the medical device comprises one or more additional sensors configured to generate one or more additional signals indicative of one or more physiological parameters of the patient, and wherein the processing circuitry is further configured to: determine that the patient is asleep based on the one or more physiological parameters; measure one or more sleep metrics while the patient is determined to be asleep based on the one or more physiological parameters; correlate the one or more sleep metrics with the one or more noise levels experienced by the patient; and determine the heart disease risk for the patient based at least in part on the correlation between the one or more sleep metrics and the one or more noise levels.

Example 13. The medical device system of any one or more of Examples 1-12, wherein the computing device is configured to emit one or more sounds having known noise levels, and wherein the processing circuitry is configured to calibrate the one or more sound sensors of the medical device based on the one or more sounds emitted from the computing device.

Example 14. The medical device system of any one or more of Examples 1-13, wherein the one or more sound sensors are a first set of one or more sound sensors, the sound signal comprises a first sound signal, and the one or more noise levels comprises a first one or more noise levels, and wherein the medical device system further comprises the computing device comprising a second set of one or more sound sensors configured to generate a second sound signal, wherein the processing circuitry is configured to: determine, based on the second sound signal, a second one or more noise levels; and calibrate the first set of one or more sound sensors based on the second one or more noise levels.

Example 15. The medical device system of any one or more of Examples 1-14, wherein the one or more sound sensors are configured to generate the sound signal continuously.

Example 16. The medical device system of Example 15, wherein the processing circuitry is configured to determine noise levels continuously based on the sound signal.

Example 17. The medical device system of Example 15, wherein the processing circuitry is configured to determine noise levels at predetermined intervals.

Example 18. The medical device system of any one or more of Examples 1-17, wherein to determine the heart disease risk based at least in part on the one or more noise levels, the processing circuitry is configured to determine whether the one or more noise levels satisfy one or more noise exposure criteria.

Example 19. The medical device system of Example 18, wherein the one or more noise exposure criteria comprise a threshold noise magnitude.

Example 20. The medical device system of Example 18 or 19, wherein the one or more noise exposure criteria comprise a threshold noise duration.

Example 21. The medical device system of any one or more of Examples 1-20, wherein the heart disease risk comprises one or more of a heart failure risk, a hypertension risk, or an arrhythmia risk.

Example 22. The medical device system of any one or more of Examples 1-21, wherein to determine the heart disease risk, the processing circuitry is configured to apply the one or more noise levels to a machine learning model.

Example 23. The medical device system of Example 22, wherein the medical device comprises one or more additional sensors configured to generate one or more additional signals indicative of one or more physiological parameters of the patient, and wherein the processing circuitry is further configured to determine one or more additional inputs for the machine learning model based on the one or more additional signals.

Example 24. The medical device system of any one or more of Examples 1-23, wherein the medical device is an implantable medical device configured for subcutaneous implantation.

Example 25. The medical device system of Example 24, wherein the implantable medical device comprises: a housing have a length from a first and to a second end, a width, and a depth, wherein the length is greater than the width, and the width is greater than the depth, and wherein the one or more sound sensors are within the housing; a first electrode at or proximate the first end of the housing; a second electrode at or proximate the second end of the housing; and circuitry within the housing, the circuitry configured to sense an electrocardiogram via the first electrode and the second electrode.

Example 26. The medical device system of any one or more of Examples 1-25, wherein the medical device comprises the processing circuitry.

Example 27. The medical device system of any one or more of Examples 1-25, wherein the processing circuitry comprises: processing circuitry of the medical device configured to determine the one more noise levels; and processing circuitry of at least one of the computing device or a cloud computing system that communicates with the computing device via a network configured to determine the heart disease risk and generate the output.

Example 28. The medical device system of any one or more of Examples 1-23, wherein the medical device comprises an insertable cardiac monitor comprising: a hermetically sealed housing configured for subcutaneous implantation within the patient, wherein the housing has a length from a first end to a second end, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm; the processing circuitry within the housing; a power source within the housing and operatively coupled to the processing circuitry; a memory within the housing and operatively coupled to the processing circuitry; sensing circuitry within the housing and operatively coupled to the processing circuitry; a first electrode at or proximate the first end of the housing and operatively coupled to the sensing circuitry; and a second electrode at or proximate the second end of the housing and operatively coupled to the sensing circuitry.

Example 29. A method comprising: determining, by processing circuitry of a medical device system including a medical device, based on sound signal generated by a sound sensor of the medical device, one or more noise levels experienced by a patient; determining, by the processing circuitry, a heart disease risk for the patient based at least in part on the one or more noise levels; and generating, by the processing circuitry, an output corresponding to the heart disease risk to a computing device of the patient or another user.

Example 30. The method of Example 29, wherein determining the one or more noise levels comprises: creating a noise profile for the patient based on the plurality of noise levels determined during the time period, wherein the noise profile comprises at least the plurality of noise levels determined and a corresponding time stamp for each of the plurality of noise levels; and determining the heart disease risk for the patient based at least in part on the noise profile.

Example 31. The method of Example 30, further comprising outputting, by the processing circuitry, the noise profile for the patient for display on the computing device.

Example 32. The method of any one or more of Examples 29-31, wherein determining the one or more noise levels comprises determining the one or more noise levels during one or more regularly occurring, predetermined time periods.

Example 33. The method of Example 32, wherein the one or more regularly occurring, predetermined time periods comprise one or both of a nighttime period and a daytime period.

Example 34. The method of Example 33, wherein determining the heart disease risk comprises: comparing the one or more noise levels during the nighttime period to a first one or more criteria; and comparing the one or more noise levels during the daytime period to a second one or more criteria that are different than the first one or more criteria.

Example 35. The method of any one or more of Examples 29-34, further comprising determining, by the processing circuitry, that an activity level of the patient is below a threshold, wherein determining the one or more noise levels comprises determining the one or more noise levels experienced by the patient based on the sound signal generated when the activity level of the patient is below the threshold.

Example 36. The method of Example 35, further comprising determining, by the processing circuitry, the activity level based on a motion signal generated by an accelerometer of the medical device.

Example 37. The method of any one or more of Examples 29-36, further comprising identifying, by the processing circuitry, one or more noise conditions causing the one or more noise levels experienced by the patient.

Example 38. The method of Example 37, wherein identifying the one or more noise conditions comprises determining locations of the patient associated with the noise levels via global positioning system data.

Example 39. The method of Example 37 or 38, wherein identifying the one or more noise conditions comprises determining times of day associated with the noise levels via a clock.

Example 40. The method of any one or more of Examples 29-39, wherein the medical device comprises one or more additional sensors configured to generate one or more additional signals indicative of one or more physiological parameters of the patient, and the method further comprises: determining, by processing circuitry, that the patient is asleep based on the one or more physiological parameters; measuring, by the processing circuitry, one or more sleep metrics while the patient is determined to be asleep based on the one or more physiological parameters; correlating, by the processing circuitry, the one or more sleep metrics with the one or more noise levels experienced by the patient; and determining, by the processing circuitry, the heart disease risk for the patient based at least in part on the correlation between the one or more sleep metrics and the one or more noise levels.

Example 41. The method of any one or more of Examples 29-40, wherein the computing device is configured to emit one or more sounds having known noise levels, the method further comprising calibrating, by the processing circuitry, the one or more sound sensors of the medical device based on the one or more sounds emitted from the computing device.

Example 42. The method of any one or more of Examples 29-41, wherein the one or more sound sensors are a first set of one or more sound sensors, the sound signal comprises a first sound signal, and the one or more noise levels comprises a first one or more noise levels, the computing device comprises a second set of one or more sound sensors configured to generate a second sound signal, and the method further comprises: determining, by the processing circuitry and based on the second sound signal, a second one or more noise levels; and calibrating, by the processing circuitry, the first set of one or more sound sensors based on the second one or more noise levels.

Example 43. The method of any one or more of Examples 29-42, wherein the one or more sound sensors are configured to generate the sound signal continuously.

Example 44. The method of Example 43, wherein determining the one or more noise levels comprises determining the one or more noise levels continuously based on the sound signal.

Example 45. The method of Example 44, wherein determining the one or more noise levels comprises determining the one or more noise levels at predetermined intervals based on the sound signal.

Example 46. The method of any one or more of Examples 29-45, wherein determining the heart disease risk based at least in part on the one or more noise levels comprises determining whether the one or more noise levels satisfy one or more noise exposure criteria.

Example 47. The method of Example 46, wherein the one or more noise exposure criteria comprise a threshold noise magnitude.

Example 48. The method of Example 46 or 47, wherein the one or more noise exposure criteria comprise a threshold noise duration.

Example 49. The method of any one or more of Examples 29-48, wherein the heart disease risk comprises one or more of a heart failure risk, a hypertension risk, or an arrhythmia risk.

Example 50. The method of any one or more of Examples 28-48, wherein determining the heart disease risk comprises applying the one or more noise levels to a machine learning model.

Example 51. The method of Example 50, wherein the medical device comprises one or more additional sensors configured to generate one or more additional signals indicative of one or more physiological parameters of the patient, the method further comprising determining, by the processing circuitry, one or more additional inputs for the machine learning model based on the one or more additional signals.

Example 52. The method of any one or more of Examples 29-51, wherein the medical device is an implantable medical device configured for subcutaneous implantation.

Example 53. The method of Example 52, wherein the implantable medical device comprises: a housing have a length from a first and to a second end, a width, and a depth, wherein the length is greater than the width, and the width is greater than the depth, and wherein the one or more sound sensors are within the housing; a first electrode at or proximate the first end of the housing; a second electrode at or proximate the second end of the housing; and circuitry within the housing, the circuitry configured to sense an electrocardiogram via the first electrode and the second electrode.

Example 54. The method of any one or more of Examples 29-53, wherein the medical device comprises the processing circuitry.

Example 55. The method of any one or more of Examples 29-54, wherein the processing circuitry comprises: processing circuitry of the medical device configured to determine the one more noise levels; and processing of at least one of the computing device or a cloud computing system that communicates with the computing device via a network configured to determine the heart disease risk and generate the output.

Example 56. A non-transitory computer readable storage medium comprising instructions that, when executed, cause processing circuitry of a medical device system to perform the method of any of Examples 19-55.

1. A medical device system comprising:
a medical device comprising one or more sound sensors configured to generate a sound signal including noise experienced by a patient; and
processing circuitry configured to:
determine, based on the sound signal, one or more noise levels experienced by a patient;
determine a heart disease risk for the patient based at least in part on the one or more noise levels; and
generate an output corresponding to the heart disease risk to a computing device of the patient or another user.

2. The medical device system of claim 1, wherein to determine the one or more noise levels, the processing circuitry is configured to determine a plurality of noise levels experienced by the patient over a time period, and wherein the processing circuitry is further configured to:
create a noise profile for the patient based on the plurality of noise levels determined during the time period, wherein the noise profile comprises at least the plurality of noise levels determined and a corresponding time stamp for each of the plurality of noise levels; and
determine the heart disease risk for the patient based at least in part on the noise profile.

3. The medical device system of claim 2, wherein the processing circuitry is further configured to output the noise profile for the patient for display on the computing device.

4. The medical device system of claim 1, wherein the one or more noise levels are determined during one or more regularly occurring, predetermined time periods.

5. The medical device system of claim 4, wherein the one or more regularly occurring, predetermined time periods comprise one or both of a nighttime period and a daytime period.

6. The medical device system of claim 5, wherein to determine the heart disease risk the processing circuitry is configured to:
compare the one or more noise levels during the nighttime period to a first one or more criteria; and
compare the one or more noise levels during the daytime period to a second one or more criteria that are different than the first one or more criteria.

7. The medical device system of claim 1, wherein the processing circuitry is further configured to:
determine that an activity level of the patient is below a threshold; and
determine the one or more noise levels experienced by the patient based on the sound signal generated when the activity level of the patient is below the threshold.

8. The medical device system of claim 7, wherein the medical device comprises an accelerometer configured to generate a motion signal of the patient, wherein the processing circuitry determines the activity level based on the motion signal.

9. The medical device system of claim 1, wherein the processing circuitry is further configured to identify one or more noise conditions causing the one or more noise levels experienced by the patient.

10. The medical device system of claim 9, wherein to identify the one or more noise conditions, the processing circuitry is further configured to determine locations of the patient associated with the noise levels via the global positioning system data.

11. The medical device system of claim 9, further comprising a clock, wherein to identify the one or more noise conditions, the processing circuitry is further configured to determine times of day associated with the noise levels via the clock.

12. The medical device system of claim 1, wherein the medical device comprises one or more additional sensors configured to generate one or more additional signals indicative of one or more physiological parameters of the patient, and wherein the processing circuitry is further configured to:
determine that the patient is asleep based on the one or more physiological parameters;
measure one or more sleep metrics while the patient is determined to be asleep based on the one or more physiological parameters;
correlate the one or more sleep metrics with the one or more noise levels experienced by the patient; and
determine the heart disease risk for the patient based at least in part on the correlation between the one or more sleep metrics and the one or more noise levels.

13. The medical device system of claim 1, wherein the computing device is configured to emit one or more sounds having known noise levels, and wherein the processing circuitry is configured to calibrate the one or more sound sensors of the medical device based on the one or more sounds emitted from the computing device.

14. The medical device system of claim 1, wherein the one or more sound sensors are a first set of one or more sound sensors, the sound signal comprises a first sound signal, and the one or more noise levels comprises a first one or more noise levels, and wherein the medical device system further comprises the computing device comprising a second set of one or more sound sensors configured to generate a second sound signal, wherein the processing circuitry is configured to:
determine, based on the second sound signal, a second one or more noise levels; and
calibrate the first set of one or more sound sensors based on the second one or more noise levels.

15. A method comprising:
determining, by processing circuitry of a medical device system including a medical device, based on sound signal generated by a sound sensor of the medical device, one or more noise levels experienced by a patient;
determining, by the processing circuitry, a heart disease risk for the patient based at least in part on the one or more noise levels; and
generating, by the processing circuitry, an output corresponding to the heart disease risk to a computing device of the patient or another user.

16. The method of claim 15, wherein determining the one or more noise levels comprises:
creating a noise profile for the patient based on the plurality of noise levels determined during the time period, wherein the noise profile comprises at least the plurality of noise levels determined and a corresponding time stamp for each of the plurality of noise levels; and
determining the heart disease risk for the patient based at least in part on the noise profile.

17. The method of claim 15, wherein determining the one or more noise levels comprises determining the one or more noise levels during one or more regularly occurring, predetermined time periods.

18. The method of claim 15, further comprising determining, by the processing circuitry, that an activity level of the patient is below a threshold, wherein determining the one or more noise levels comprises determining the one or more noise levels experienced by the patient based on the sound signal generated when the activity level of the patient is below the threshold.

19. The method of claim 15, further comprising identifying, by the processing circuitry, one or more noise conditions causing the one or more noise levels experienced by the patient.

20. A non-transitory computer readable storage medium comprising instructions that, when executed, cause processing circuitry of a medical device system including a medical device to perform:
determining, based on sound signal generated by a sound sensor of the medical device, one or more noise levels experienced by a patient;
determining a heart disease risk for the patient based at least in part on the one or more noise levels; and
generating an output corresponding to the heart disease risk to a computing device of the patient or another user.
